(19) United States
(12) Patent Application Publication
(10) Pub. No.: US 2021/0138238 A1
(43) Pub. Date: May 13, 2021

(54) NERVE STIMULATION AND MONITORING DEVICE

(71) Applicants: GALVANI BIOELECTRONICS LIMITED, Brentford (GB); UCL BUSINESS LTD, London (GB)

(72) Inventors: David Holder, London, Greater London (GB); Kirill ARISTOVICH, London, Greater London (GB); Daniel CHEW, Brentford (GB); Matteo DONEGA, Brentford (GB)

(21) Appl. No.: 16/622,679

(22) PCT Filed: Jun. 22, 2018
(86) PCT No.: PCT/GB2018/051749

§ 371 Date: Dec. 13, 2019

Related U.S. Application Data
(60) Provisional application No. 62/609,227, filed on Dec. 21, 2017.

(30) Foreign Application Priority Data
Jun. 22, 2017 (GB) 1710026.4

(51) Int. Cl. A61N 1/05; A61N 1/36

(52) U.S. Cl. CPC A61N 1/0556; A61N 1/36175; A61N 1/36171; A61N 1/36157

(57) ABSTRACT

There is provided a nerve interface device including (1, 3) comprising: at least one cuff portion; and a first pair of electrodes mounted on the at least one cuff portion. The cuff portion has an assembled position in which the cuff portion forms at least part of a passageway for receiving a nerve along a cuff axis passing through the passageway. The electrodes of the first pair are spaced apart from one another in the direction of the cuff axis.

### PRIORITY CLAIM

The present application is a National Phase entry of PCT Application No. PCT/GB2018/051749, filed Jun. 22, 2018, which claims priority from GB Patent Application No. 1710026.4, filed Jun. 22, 2017, and U.S. Patent Provisional Application No. 62/609,227, filed Dec. 21, 2017, which is hereby fully incorporated herein by reference.

### TECHNICAL FIELD

This disclosure relates to a system, a method and a computer program for stimulating a nerve and for detecting nerve activity in a human or an animal subject.

### BACKGROUND

It is desirable to be able to identify bundles of nerves, within a complex nerve, which are specific to certain organs. The vagus nerve is an example of a complex nerve. In addition, it is desirable to be able to selectively stimulate specific nerves bundles. This may allow certain responses in specific organs to be induced.

One known method that may allow for the identification of organ specific fibers within a peripheral nerve involves inserting an electrode array with penetrating shanks into the nerve and recording local field potentials. The correlation of the recording of spontaneous local field potentials with physiological activity, such as ECG and respiration, allows the position of organ specific bundles to be determined. This known method has drawbacks because the insertion of electrodes into the nerve may result in the damage of fibers. This has potentially serious consequences.

Fast Neural Electrical Impedance Tomography (EIT) is a known method for producing tomographic 3D images of neuronal function. WO 2016/170327 describes one example of a device that can be used for monitoring the activity of nerves using EIT. However, in some scenarios it is difficult to associate nerve activity detected using EIT with physiological activity occurring in a subject. This is particularly difficult for spontaneous nerve activity occurring a complex nerve.

Selective stimulation of specific fiber types within a mixed nerve (including myelinated and unmyelinated fibers) could provide higher specificity and lower side effects when targeting specific types of fibers to cause specific physiological responses. However, this can be difficult to achieve with known electrodes assemblies, such as the electrode ring described in WO 2016/170327. Furthermore, selective stimulation using penetrative electrodes is undesirable as outlined above.

Therefore, there exists a need for a way in which to selectively stimulate and monitor nerves more accurately.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one aspect of the disclosure there is provided a nerve interface device comprising: at least one cuff portion; and a first pair of electrodes mounted on the at least one cuff portion; wherein the cuff portion has an assembled position in which the cuff portion forms at least part of a passageway for receiving a nerve along a cuff axis passing through the passageway; and wherein electrodes of the first pair are spaced apart from one another in the direction of the cuff axis.

In another aspect of the disclosure there is provided a nerve stimulation system comprising: a first nerve interface device as described herein; and a stimulation device arranged to generated an electrical signal; wherein the stimulation device is arranged for electrical communication with the first pair of electrodes or each of the plurality of pairs of electrodes of the first device, to provide the electrical signal to the first pair or the plurality of pairs of electrodes of the first device.

In another aspect of the disclosure there is provided a nerve stimulation method comprising: providing a first nerve interface device as described herein; and stimulating the first pair of electrodes or each of the plurality of pairs of electrodes of the first device, to provide an electrical signal to the first pair or the plurality of pairs of electrodes of the first device.

In another aspect of the disclosure there is provided a computer program comprising code portions which when loaded and run on a computing device cause the computing device to: stimulate the first pair of electrodes or each of the plurality of pairs of electrodes of the device described herein, to provide an electrical signal to the first pair or the plurality of pairs of electrodes of the first device.

In another aspect of the disclosure there is provided an implantable system for stimulating and/or monitoring activity in a nerve comprising: at least one nerve interface device as described herein arranged, in use, to apply an electrical signal to at least one nerve fiber of a subject; a signal generator configured to generate a signal to be delivered to the at least one nerve fiber by the first pair of electrodes to modulate neural activity within the at least one nerve fiber; a control sub-system configured to cause the signal generator to deliver the signal to the first pair of electrodes; wherein the control sub-system is configured to cause the signal generator to deliver the signal to the first pair of electrodes either a) upon receiving a trigger generated by an operator; or b) according to a pre-determined pattern.

In another aspect of the disclosure there is provided a method of modulating activity in at least one nerve fiber of a subject, comprising: providing an implantable system as described herein; causing the signal generator to deliver a signal to the first pair of electrodes; and delivering the signal via the first pair of electrodes to the at least one nerve fiber.

In another aspect of the disclosure there is provided an implantable system for stimulating and monitoring activity in a nerve comprising: first and second nerve interface devices, as described herein, the first arranged, in use, to apply an electrical signal to at least one nerve fiber of a subject and the second arranged, in use, to detect said electrical signal in the at least one nerve fiber; a signal generator configured to generate a signal to be delivered to the at least one nerve fiber by the first pair of electrodes in the first nerve interface device to modulate neural activity within the at least one nerve fiber; a control sub-system configured to cause the signal generator to deliver the signal to the first pair of electrodes in the first nerve interface device; a detection sub-system configured to detect activity within the at least one nerve fiber at the first pair of electrodes in the second nerve interface device.

In another aspect of the disclosure there is provided a method of stimulating and monitoring activity in at least one nerve fiber of a subject, comprising: providing an implantable system as described here; causing the signal generator to deliver a signal to the first pair of electrodes in the first nerve interface device; and detecting via the first pair of electrodes in the second nerve interface device activity in the nerve, the activity caused by the signal delivered to the at least one nerve fiber by the first pair of electrodes in the first nerve interface device.

In another aspect of the disclosure there is provided an implantable device comprising a system as described herein.

In another aspect of the disclosure there is provided a computer-readable having stored thereon a computer program as described herein.

### BRIEF DESCRIPTION OF THE DRAWINGS

Embodiments of the disclosure will be described, by way of example, with reference to the following drawings, in which:

FIG. 1 illustrates examples of a nerve stimulation device;

FIG. 2 illustrates schematic representations of the nerve stimulation devices;

FIG. 3 illustrates measurements of compound action potentials (CAP) measured in response to stimulation of a nerve using the nerve stimulation devices;

FIG. 4 illustrates further measurements of CAP measured in response to stimulation of a nerve using the nerve stimulation devices;

FIG. 5 illustrates the position of electrode pairs in the nerve stimulation devices;

FIGS. 6A and 6B illustrate measurements of physiological activity and CAP measured in response to stimulation of a nerve using the nerve stimulation devices;

FIG. 7 illustrates images of nerve activity; and

FIG. 8 illustrates an overview of the system.

### DETAILED DESCRIPTION

Described herein is a device, system and method that allows specific nerve fibers to be selectively stimulated within a complex nerve such as the vagus nerve. This enables fibers to be targeted more precisely.

For example, specific stimulation of pulmonary bundles of the vagus nerve could help treat asthma and other respiratory conditions, whilst avoiding side-effects on other organs. Alternatively, selective stimulation of descending c-fiber bundles could optimize the stimulation of visceral organs, without affecting the cardio-respiratory system. Also, selective stimulation could be used to avoid contraction of the thyroarytenoid (TA) muscle of the larynx, which is the most common and serious side-effect of current vagus nerve stimulators used to treat inflammatory diseases. In addition, the combination of EIT and selective stimulation maybe provided in a ‘closed-loop’ system to regulate the cardio-respiratory system and/or visceral organ functions. This system may be provided in an implantable device.

Referring to FIG. 1, there is provided a first nerve stimulation device 1 (otherwise referred to as electrode array “A”) and a second nerve stimulation device 3 (otherwise referred to as electrode array “B”). Each one of the arrays 1, 3 comprises a cuff portion 5, 7 upon which is provided a plurality of electrodes 9, 11.

The cuff portion 5, 7 is a flexible sheet with the electrodes 9, 11 mounted on the sheet. The sheet can be wrapped around a nerve of a subject 13, such that the electrodes 9, 11 form an electrical contact with the nerve at various points around the surface of the nerve 13. When the cuff is wrapped around the nerve 11, in its assembled position, the cuff forms an aperture (or tubular section/passageway) for receiving the nerve 13. As illustrated, the cuff 5, 7 receives the nerve along a cuff axis 19 (or longitudinal axis) which passes through the middle of the cuff 5, 7. This cuff axis 19 is also the longitudinal axis of the nerve 13.

As illustrated, the arrays 1, 3 can be separated from one another along the length of the nerve 13. In this example, the arrays 1, 3 are separated by a distance of 40 mm.

The electrodes may comprise stainless steel and can be fabricated by laser cutting the electrodes into a film. In one example, the film comprises silicon.

As illustrated in the expanded cross-sectional view 23, the aperture formed by the cuff 7 has a diameter (d1). In one example, d1 is 2.8 mm. The cuff axis 19 is perpendicular to the diameter and parallel with the depth of the aperture. In other words, the cuff axis is parallel with the depth of the tubular section. Furthermore, the pair of electrodes are offset from one another in a direction perpendicular to the diameter of the aperture and parallel with the depth of the aperture.

Each one of the arrays 1, 3 comprises a plurality of pairs of electrodes 15, 17. These electrode pairs 15, 17 are offset, or spaced apart, from one another in the direction of the cuff axis 19. Thus, the stimulation device can apply a signal to an electrode pair 15, 17 and induce a signal between the electrodes in the pair 15, 17 in a longitudinal direction along the nerve 11. In this way, an electrical channel is provided in the direction of the longitudinal axis 19 of the nerve. This can be used to stimulate specific nerve fibers 21 in the nerve 13, which may be associated with specific organs or physiological responses in the subject. In some embodiments, the electrodes in each pair are offset from one another by a distance of between 0.5 mm and 4.5 mm in the longitudinal direction.

In this example, the plurality of electrodes in each array 1, 3 are mounted on the same cuff 5, 7. However, it may be possible to provide more than one cuff portion, with some electrode(s) provided on one cuff portion and some electrode(s) provided on another cuff portion.

Each one of the arrays 1, 3 comprises a first set of electrodes 25, 29 and a second set of electrodes 27, 31 mounted on the cuff portion. In the assembled position, the electrodes of first set of electrodes 25, 29 are mounted offset from one another in a direction perpendicular to the cuff axis; and the electrodes of second set of electrodes 27, 31 are mounted offset from one another in a direction perpendicular to the cuff axis 19. As illustrated, the electrodes of the first set of electrodes 25, 29 and the second set of electrodes 27, 31 are spaced in a ring around a circumference of the cuff 5, 7. Each one of the rings of electrodes may have a diameter that is perpendicular to the cuff axis 19 and that is perpendicular to the diameter of the cuff, d1. Alternatively, each one of the rings of electrodes may have any another suitable angular offset from the cuff axis 19 and/or the cuff diameter, d1. Therefore, each one of the rings may be positioned at a slant such that the electrodes on a first side of the ring are offset from the electrodes on a second opposing side of the ring in the longitudinal direction. The electrodes in each of the first set 25, 29 or the second set 27, 32 of electrodes may be arranged in a spiral, or a helical configuration within each set. In the helical configuration, the electrodes in each pair may be offset from one another by a distance greater than 4.5 mm in the longitudinal direction. For instance, the electrodes may be offset from one another by a distance of up to 15 mm.

The electrodes of the first set of electrodes 25, 29 comprise a first electrode in a pair of electrodes 15, 17, and the electrodes of the second set of electrodes 27, 31 comprise a second electrode in the pair 15, 17. The electrodes in each pair 15, 17 are offset from one another along the length of the nerve 11.

In each array 1, 3 the first set 25, 29 and/or the second set 27, 31 of electrodes may comprise 4 to 96 electrodes. In various embodiments, the first set 25, 29 and/or the second set 27, 31 of electrodes comprise 4 to 18 electrodes. It has been found that this number of electrodes is more suitable for use with the vagus nerve of a pig and a sheep, and therefore it is expected that this number of electrodes is more suitable for use with humans.

In some embodiments, the maximum number of electrodes in each ring is defined by the following equation:

N=(2rrR/2)/D in which:

N=the number of electrodes

R=the radius of the nerve with which the device is to be used

D=the average diameter of the fascicles within the nerve.

Therefore, the maximum number of electrodes in each ring may be half the circumference of the nerve with which the device is to be used divided by the average diameter of the fascicle within the nerve. The result of this calculation can be rounded up or down to the nearest integer. In some embodiments, the result of the calculation is rounded down to the nearest integer. The radius of the nerve is approximately equal to the radius of the cuff in its assembled position.

As an example, the vagus nerve in sheep, and humans, has a diameter of around 2-4 mm. In sheep, for example, the diameter is about 2.5 mm, and the average fascicle diameter is about 0.25 mm. Using the above equation the maximum number of electrodes should be around 15 electrodes for optimal selectivity. The number of electrodes can then be adjusted depending on the size of the area of the nerve controlling the physiological function of interest.

The number of electrodes calculated using the above equation can provide maximum selectivity. Any more electrodes than the maximum number of electrodes calculated may be redundant, as they are likely to be smaller than the actual diameter of each nerve fascicle (i.e. more than one electrode will activate the same nerve fascicle). Using the maximum number of electrodes provides greater selectivity and efficiency.

In some embodiments, each electrode has a surface of between 0.5 mm and 2 mm in length and between 0.05 mm and 2 mm in width. In some embodiments, the width of the surface of each electrode is selected based on the diameter of a nerve fascicle, or on the diameters of a group of nerve fascicles, with which the device is to be used. For instance, the width of the surface of each electrode is selected so that at least it is the same as the diameter of a nerve fascicle, or average the diameter of a group of nerve fascicles, with which the device is to be used. Smaller widths may result in redundant electrodes. This allows the electrodes to target the desired fascicles more accurately, while reducing the number of electrodes required.

In a specific example illustrated in FIG. 2, the first set of electrodes 25 and the second set of electrodes 27 of the first array 1 comprises 14 electrodes. Also, the first set of electrodes 25 and the second set of electrodes 27 of the second array 3 comprises 14 electrodes. As illustrated, each set of electrodes 25, 27, 29, 31 comprises a plurality of electrodes arranged sequentially to form a straight line of electrodes on the cuff sheet.

FIG. 2 illustrates two schematic views of each of the electrode arrays 1, 3. Each of the electrodes in the arrays 1, 3 has a surface for making electrical contact with the nerve 13. In the first array 1, this surface is rectangular with a width of 0.2 mm and a length of 3 mm. In the second array 3, the surface is also rectangular with a width of 0.2 mm and a length of 1 mm. In another example array (not shown), each of the electrodes has a square surface. This square surface may be 0.2 mm wide and 0.2 mm long.

In each of the arrays 1, 3 illustrated in FIG. 2, the electrodes are paired. Each electrode in the first set 25, 29 is paired with an opposing electrode in the second set 27, 31. In the example illustrated, the electrodes in each pair are offset from one another by a distance of 3 mm. Thus, the first set of electrodes 25, 29 is offset from the second set of electrodes 29, 31 by a distance of 3 mm. This distance is measured in the direction of the cuff axis 19.

It will be appreciated that other distances between pairs/sets of electrodes could be used. For instance, the electrode pairs/sets may be offset from one another by a distance of 2 mm. In another example, the electrode pairs/sets may be offset from one another by a distance of 1 mm.

One or more of the arrays 1, 3 may be provided in a nerve stimulation system comprising a stimulation device arranged to generate an electrical signal. In this example, the stimulation device is arranged for electrical communication with the first pair of electrodes 15, 17 or each of the plurality of pairs of electrodes of the first device. In this way, the stimulation device can provide an electrical signal to pairs of electrodes.

The stimulation device is capable of generating electrical signals with a variety of different properties. For example, the stimulation device may be arranged to generate signals each with a different pulse duration, frequency, pulse width and current. In addition, the stimulation device may be capable of generating a bipolar pulse.

In one example, the signal has a pulse duration of 1 ms. The signal may have a frequency of 1-50 Hz frequency. More specifically, the signal may have a frequency of 2 Hz. The signal may have a pulse width of 50-1000 μs. The amplitude of the current of the signal may be between 100-2000 mA.

In another example, the signal has a current of 500 μm, a duration of 0.1 ms and/or a frequency of 5 Hz. In yet another example, the signal has a frequency of 20 Hz and/or a duration of 60 seconds.

The system may also comprise a physiological sensor arranged to detect physiological activity in a subject. This sensor may be used to detect activity in the subject such as heart rate. The system may also comprise a comparison module arranged to detect a relationship between the electrical signal and physiological activity. Thus, relationships between nerve activity and physiological activity can be determined.

In one example application, the electrodes of the arrays are placed on the right vagus nerve of anesthetized adult sheep and stimulation is applied between electrode pairs. In this example, the arrays are arranged in a similar fashion to that illustrated in FIG. 1 with the nerve 13 being the vagus nerve of the sheep.

FIG. 3 illustrates a number of charts which show the response induced in the nerve 13 when stimulation was applied to the electrode pairs. Charts 35 and 37 illustrate the compound action potential (CAP) measured in the nerve of different sheep when stimulation was applied to electrode pairs of the second array 2. On the other hand, charts 39 and 41 illustrate the CAP measured in the nerve of different sheep when stimulation was applied to electrode pairs of the second array 2.

It was found that in any of the electrode pairs of the second array 3, the 1 mm long electrodes mostly elicited fast fiber response (myelinated fibers). In addition, it was found that the longer electrode arrays of the first array 1 stimulated both slow (unmyelinated) and fast fibers, but with a much higher proportion of slow fibers (unmyelinated) being stimulated. This was found when either the same current or the same charge density was applied in either one of the electrode arrays.

Furthermore, it was found that the first array 1 was able to reliably cause bradypnea (slow breathing) when stimulating the vagus nerve. On the other hand, the second array 2 always failed to achieve this (with any of the tested combination of electrodes) even at much higher charge densities.

The arrays described above can be used in order to selectively stimulate specific nerve fibers in a nerve. Referring to FIG. 4, arrays comprising two electrode rings each comprising 14 electrodes were used to selectively stimulate nerve fibers. Here, each electrode had a surface of 0.2 mm in width and 0.2 mm in length, and each pair of electrodes were 1 mm apart. One such array 43, was positioned on the vagus nerve 13 of a subject in order to provide selective stimulation to the nerve.

A stimulation device was used to generate electrical signals. In this example, the signals comprise bipolar stimulating pulses with a current of 500 μA, a duration of 0.1 ms and a frequency of 5 Hz. These signals were applied to electrode pairs, one longitudinal pair at a time. CAP responses to the stimulation were measured using a cortec array 47 placed on the pulmonary branch 13′ of the nerve 13 and another cortec array 45 placed on the rest of descending vagus nerve fibers 13″.

The activation patterns for each of the 14 pairs of electrodes are illustrated in the chart 49. In the charts 49 the lines represent the readings from the pulmonary branch and the readings from the rest of vagus nerve fibers.

As illustrated, it can be seen that there was a significant difference in the activation patterns depending on the pairs of electrodes being stimulated at a particular time. Therefore, it will be appreciated that the electrode array 43 is capable of selectively stimulating nerve fibers in a nerve.

Referring again to FIG. 1, another example of selective stimulation will be described. In this example, an in-vivo experiment was conducted in which selective stimulation was combined with EIT imaging. Two arrays 1, 3 were implanted on the right cervical vagus nerve 13 of an anesthetized sheep. The first array 1 (Array A) was used to stimulate the nerve 13, whilst the second array 3 (Array B) was used for CAP recording and EIT imaging. The arrays 1, 3 were placed 40 mm apart. In addition, physiological sensors were used to measure physiological parameters, such as end tidal CO2 (EtCO2), electrocardiogram (ECG), blood pressure (BP), heart rate (HR), respiration rate (RR) and peripheral capillary oxygen saturation (SpO2) in the subject. The specific electrode arrays described above with reference to FIG. 2 were used in this example.

One longitudinal pair at a time was stimulated with 20 Hz, 0.05 ms, 20 Hz, biphasic stimulation pulses lasting 60 seconds. This was followed by rest period lasting another 60 seconds. Then, the adjacent pair of electrodes in the array was selected and the protocol repeated for all of the electrodes. The position of each of the electrode pairs is illustrated schematically in FIG. 5, in which the solid circle represents the position of the electrode pair relative to the other pairs.

The process of stimulating the electrode pairs lasted 28 minutes during which RR, BP, EtCO2, SpO2 and ECG were constantly monitored. The results of this process are illustrated in FIG. 6A and FIG. 6B in which the upper chart 51 for each pair shows physiological data and the lower chart 53 for each pair shows the average CAP measured during 60 s of stimulation.

In the upper charts 51 showing physiological data the line 55 shows HR, the line 57 shows BP and the dark line 59 shows EtCO2 indicative of breathing pattern. The line 61 shows HR measured from ECG; however, the HR from ECG readings tended to be inconsistent and, thus, will be ignored for the purposes of this example.

As illustrated in the charts 51, stimulation of specific pairs of electrodes can induce specific physiological responses. For example, stimulation of pairs 3 and 4 resulted in a change in HR and blood pressure. As another example, stimulation of pairs 10-12 resulted in a changed in breathing pattern. In this way, it is possible to determine that specific nerve fibers in proximity to the electrodes of a particular pair are associated with specific organs and physiological responses.

After selective stimulation process, a first pair of electrodes which provided the most prominent pulmonary response was selected. Then, another 3 pairs were selected: the pair opposite the first pair, the pair located 90° clockwise of the first pair and the pair located 90° anti-clockwise of the first pair. This resulted in the selection of 4 pairs, each located at 4 equidistant points around the circumference of the array. Then, by stimulating 1 pair at a time, full EIT recording was performed using the opposite array. In this example, a 14-pair injecting protocol was used with 30 seconds per injection for EIT recording. This required 7 mins per imaging data set. The EIT signal used has a frequency of 6 kHz and 9 kHz, with a current amplitude of 100 uA.m Thus, when EIT was combined with stimulation of the most respiratory effective pair of electrodes and the opposing pair, different areas for the vagus nerve were imaged. The results of the EIT imaging process are illustrated in FIG. 7.

Referring to FIG. 7, the images show EIT imaging reconstruction obtained in two different sheep when selective stimulation was performed with array B, and EIT recording was performed with array A. The images in the first column 63 show the EIT images obtained during stimulation of an electrode pair that was found not to cause any respiratory change. The images in the second column 65 show the EIT images obtained during stimulation of an electrode pair that was found to cause respiratory changes. Therefore, it has been shown that the electrode arrays described herein allow specific nerve fibers to be selectively stimulated and imaged.

In another example, an implantable system for stimulating and/or monitoring activity in a nerve is provided. This system includes at least one nerve interface device, which may correspond with one or more of the nerve interface device described above. The at least one nerve interface device is arranged, in use, to apply an electrical signal to at least one nerve fiber of a subject. The electrical signal may be applied in a manner consistent with that described above.

The implantable system may comprise a signal generator which is configured to generate a signal to be delivered to the at least one nerve fiber by the first pair of electrodes of the nerve interface device to modulate neural activity within the at least one nerve fiber. The implantable system may also comprise a control sub-system configured to cause the signal generator to deliver the signal to the first pair of electrodes.

The control sub-system may be configured to cause the signal generator to deliver the signal to the first pair of electrodes upon receiving a trigger generated by an operator. In addition, or as an alternative, the control sub-system may be configured to cause the signal generator to deliver the signal to the first pair of electrodes according to a pre-determined pattern.

The implantable system may further comprises a detection sub-system configured to detect activity within the at least one nerve fiber at the first pair of electrodes. In this way, the system is able to monitor activity in the nerve, for instance, via imaging the nerve using a technique such as EIT.

The implantable system may be further configured to generate probe electrical signals to be delivered to the at least one nerve fiber by the first pair of electrodes to cause a corresponding electrical response within the at least one nerve fiber. The system may further comprise: a stimulation sub-system configured to cause the signal generator to deliver the probe electrical signals to the first pair of electrodes. The detection sub-system may be configured to detect an electrical response within the at least one nerve fiber at the first pair of electrodes.

The implantable system may further comprise one or more physiological sensors configured to detect physiological activity that is associated with corresponding neural activity within the at least one nerve fiber. An example of a physiological sensor is an ECG monitor, which can be used to monitor heart activity. In one example, the neural activity is autonomic neural activity. In particular, the detection sub-system is configured to detect the corresponding neural activity within the at least one nerve fiber at the first pair of electrodes.

The implantable system discussed herein may comprise at least one nerve interface device. Examples of nerve interface devices are described above. The stimulation sub-system may be configured to generate probe electrical signals to be delivered to the at least one nerve fiber by each of the plurality of pairs of electrodes of the nerve interface device.

The implantable system may comprise processing means configured to determine, based on the electrical responses and/or corresponding neural activity detected by the detection subsystem, electrical properties at one or more locations within the nerve fiber.

The control sub-system may be configured to determine one or more pairs of electrodes for delivering the signal based on the one or more locations within the nerve fiber at which the detection subsystem determined the electrical properties.

There is also provided a method of modulating activity in at least one nerve fiber of a subject which uses the system described herein. In the method, the system causes the signal generator to deliver a signal to the first pair of electrodes. Then, the signal is delivered via the first pair of electrodes to the at least one nerve fiber. In one example, the signal generator may be caused to deliver the signal upon receipt of a trigger signal generated by an operator. In another example, the signal generator may be caused to deliver the signal according to a predetermined pattern.

The method may further comprise the step of detecting, via the first pair of electrodes, activity in the nerve. The method may further comprise the step of delivering a probe electrical signal to the nerve via the first pair of electrodes, wherein the activity in the nerve that is detected via the first pair of electrodes is an electrical response caused by the probe electrical signal. The activity in the nerve that is detected via the first pair of electrodes may be neural activity caused by corresponding physiological activity.

In another example, there is an implantable system for stimulating and monitoring activity in a nerve. This system may comprise first and second nerve interface devices, which may be any one the devices described above. The first device may be arranged, in use, to apply an electrical signal to at least one nerve fiber of a subject. In addition, the second device may be arranged, in use, to detect said electrical signal in the at least one nerve fiber.

The system may further comprise a signal generator configured to generate a signal to be delivered to the at least one nerve fiber by the first pair of electrodes in the first nerve interface device to modulate neural activity within the at least one nerve fiber; a control sub-system configured to cause the signal generator to deliver the signal to the first pair of electrodes in the first nerve interface device; and a detection sub-system configured to detect activity within the at least one nerve fiber at the first pair of electrodes in the second nerve interface device.

In another example, there is a method of stimulating and monitoring activity in at least one nerve fiber of a subject. The method may use an implantable system, which may be one of the systems described above. The method may comprise the steps of causing the signal generator to deliver a signal to the first pair of electrodes in the first nerve interface device; and detecting via the first pair of electrodes in the second nerve interface device activity in the nerve, the activity caused by the signal delivered to the at least one nerve fiber by the first pair of electrodes in the first nerve interface device.

### An Implantable Device/System for Implementing Embodiments of the Disclosure

An implantable system according to the disclosure comprises an implantable device (e.g. implantable device 106 of FIG. 8). The implantable device comprises at least one neural interfacing element such as a transducer, for example an electrode (e.g. electrode 108), suitable for placement on, in, or around a nerve. The implantable system also can comprise a processor (e.g. microprocessor 113) coupled to the at least one neural interfacing element.

The at least one neural interfacing element may take many forms, and includes any component which, when used in an implantable device or system for implementing embodiments of the disclosure, is capable of applying a stimulus or other signal that modulates electrical activity in a nerve.

The various components of the implantable system can be part of a single physical device, either sharing a common housing or being a physically separated collection of interconnected components connected by electrical leads (e.g. leads 107). As an alternative, however, embodiments may use a system in which the components are physically separate, and communicate wirelessly. Thus, for instance, the at least one neural interfacing element (e.g. electrode 108) and the implantable device (e.g. implantable device 106) can be part of a unitary device, or together may form an implantable system (e.g. implantable system 116). In both cases, further components may also be present to form a larger device or system (e.g. system 100).

Suitable Forms of a Modulating Signal

Embodiments can use a signal applied via one or more neural interfacing elements (e.g. electrode 108) placed in signaling contact with a nerve.

Signals applied according to the disclosure are ideally non-destructive. As used herein, a “non-destructive signal” is a signal that, when applied, does not irreversibly damage the underlying neural signal conduction ability of the nerve. That is, application of a non-destructive signal maintains the ability of the nerve (e.g. a nerve) or fibers thereof, or other nerve tissue to which the signal is applied, to conduct action potentials when application of the signal ceases, even if that conduction is in practice artificially stimulated as a result of application of the non-destructive signal.

The signal will usually be an electrical signal, which may be, for example, a voltage or current waveform. The at least one neural interfacing element (e.g. electrode 108) of the implantable system (e.g. implantable system 116) is configured to apply the electrical signals to a nerve, or a part thereof. However, electrical signals are just one way of implementing embodiments of the disclosure, as is further discussed below.

An electrical signal can take various forms, for example, a voltage or current. In certain such embodiments the signal applied comprises a direct current (DC), such as a charge balanced direct current, or an alternating current (AC) waveform, or both a DC and an AC waveform. A combination of charge balanced DC and AC is particularly useful, with the DC being applied for a short initial period after which only AC is used. As used herein, “charge-balanced” in relation to a DC current is taken to mean that the positive or negative charge introduced into any system (e.g. a nerve) as a result of a DC current being applied is balanced by the introduction of the opposite charge in order to achieve overall (net) neutrality. In other words, a charge-balance DC current includes a cathodic pulse and an anodic pulse.

In certain embodiments, the DC waveform or AC waveform may be a square, sinusoidal, triangular, trapezoidal, quasitrapezodial or complex waveform. The DC waveform may alternatively be a constant amplitude waveform. In certain embodiments the electrical signal is an AC sinusoidal waveform. In other embodiments, a waveform comprises one or more pulse trains, each comprising a plurality of charge-balanced biphasic pulses.

The signal may be applied in bursts. The range of burst durations may be from seconds to hours; applied continuously in a duty cycled manner from 0.01% to 100%, with a predetermined time interval between bursts. The electric signal may be applied as step change or as a ramp change in current or intensity. Particular signal parameters for modulating (e.g. stimulating) a nerve are further described below.

Modulation of the neural activity of the nerve can be achieved using electrical signals which serve to replicate the normal neural activity of the nerve.

Signal Parameters for Modulating Neural Activity

In all of the above examples, a signal generator may be configured to deliver an electrical signal for modulating (e.g. stimulating) a nerve (e.g. the ICN). In the present application, the signal generator is configured to apply an electrical signal with certain signal parameters to modulate (e.g. stimulate) neural activity in a nerve (e.g. the ICN). Signal parameters for modulating (e.g. stimulating) the nerve, which are described herein, may include waveform, amplitude and frequency.

It will be appreciated by the skilled person that the current amplitude of an applied electrical signal necessary to achieve the intended modulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

Electrodes

As mentioned above, the implantable system comprises at least one neural interfacing element, and the neural interfacing element can be an electrode 108. The neural interface is configured to at least partially and in some embodiments fully circumvent the nerve. The geometry of the neural interface is defined in part by the anatomy of the nerve.

In some embodiments (for example, FIG. 8), electrode 108 may be coupled to implantable device 106 of implantable system 116 via electrical leads 107. Alternatively, implantable device 106 may be directly integrated with the electrode 108 without leads. In any case, implantable device 106 may comprise DC current blocking output circuits, optionally based on capacitors and/or inductors, on all output channels (e.g. outputs to the electrode 108, or physiological sensor 111). Electrode 108 may be shaped as one of: a rectangle, an oval, an ellipsoid, a rod, a straight wire, a curved wire, a helically wound wire, a barb, a hook, or a cuff. In addition to electrode 108 which, in use, is located on, in, or near a nerve (e.g. the ICN), there may also be a larger indifferent electrode placed 119 (not shown) in the adjacent tissue.

In some embodiments, electrode 108 may contain at least two electrically conductive exposed contacts 109 configured, in use, to be placed on, in, or near a nerve. Exposed contacts 109 may be positioned, in use, transversely along the axis of a nerve.

Microprocessor

The implantable system 116, in particular the implantable device 106, may comprise a processor, for example microprocessor 113. Microprocessor 113 may be responsible for triggering the beginning and/or end of the signals delivered to the nerve (e.g., a nerve) by the at least one neural interfacing element. Optionally, microprocessor 113 may also be responsible for generating and/or controlling the parameters of the signal.

Microprocessor 113 may be configured to operate in an open-loop fashion, wherein a pre-defined signal (e.g. as described above) is delivered to the nerve at a given periodicity (or 5 continuously) and for a given duration (or indefinitely) with or without an external trigger, and without any control or feedback mechanism. Alternatively, microprocessor 113 may be configured to operate in a closed-loop fashion, wherein a signal is applied based on a control or feedback mechanism. As described elsewhere herein, the external trigger may be an external controller 101 operable by the operator to initiate delivery of a signal.

Microprocessor 113 of the implantable system 116, in particular of the implantable device 106, may be constructed so as to generate, in use, a preconfigured and/or operator-selectable signal that is independent of any input. In some embodiments, however, microprocessor 113 is responsive to an external signal, for example information (e.g. data) pertaining to one or more physiological parameters of the subject.

Microprocessor 113 may be triggered upon receipt of a signal generated by an operator, such as a physician or the subject in which the device 116 is implanted. To that end, the implantable system 116 may be part of a system which additionally comprises an external system 118 comprising a controller 101. An example of such a system is described below with reference to FIG. 8.

External system 118 of system 100 is external the implantable system 116 and external to the subject, and comprises controller 101. Controller 101 may be used for controlling and/or externally powering implantable system 116. To this end, controller 101 may comprise a powering unit 102 and/or a programming unit 103. The external system 118 may further comprise a power transmission antenna 104 and a data transmission antenna 105, as further described below.

The controller 101 and/or microprocessor 113 may be configured to apply any one or more of the above signals to the nerve intermittently or continuously. Intermittent application of a signal involves applying the signal in an (on-off) n pattern, where n>1. For instance, the signal can be applied continuously for at least 5 days, optionally at least 7 days, before ceasing for a period (e.g. 1 day, 2 days, 3 days, 1 week, 2 weeks, 1 month), before being again applied continuously for at least 5 days, etc. Thus the signal is applied for a first time period, then stopped for a second time period, then reapplied for a third time period, then stopped for a fourth time period, etc. In such an embodiment, the first, second, third and fourth periods run sequentially and consecutively. The duration of the first, second, third and fourth time periods is independently selected. That is, the duration of each time period may be the same or different to any of the other time periods. In certain such embodiments, the duration of each of the first, second, third and fourth time periods may be any time from 1 second (s) to 10 days (d), 2 s to 7 d, 3 s to 4 d, 5 s to 24 hours (24 h), 30 s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5 s, 10 s, 30 s, 60 s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 d, 3 d, 4 d, 5 d, 6 d, 7 d.

In certain embodiments, the signal is applied by controller 101 and/or microprocessor for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. In certain alternative such embodiments, the signal may be applied discontinuously across the day, provided the total time of application amounts to the specified time.

Continuous application may continue indefinitely, e.g. permanently. Alternatively, the continuous application may be for a minimum period, for example the signal may be continuously applied for at least 5 days, or at least 7 days.

Whether the signal applied to the nerve is controlled by controller 101, or whether the signal is continuously applied directly by microprocessor 113, although the signal might be a series of pulses, the gaps between those pulses do not mean the signal is not continuously applied.

In certain embodiments, the signal is applied only when the subject is in a specific state e.g. only when the subject is awake, only when the subject is asleep, prior to and/or after the ingestion of food, prior to and/or after the subject undertakes exercise, etc.

The various embodiments for timing for modulation of neural activity in the nerve can all be achieved using controller 101 in a device/system of the disclosure.

Other Components of the System Including the Implantable Device

In addition to the aforementioned electrode 108 and microprocessor 113, the implantable system 116 may comprise one or more of the following components: implantable transceiver 110; physiological sensor 111; power source 112; memory 114; and physiological data processing module 115. Additionally or alternatively, the physiological sensor 111; memory 114; and physiological data processing module 115 may be part of a sub-system external to the implantable system. Optionally, the external sub-system may be capable of communicating with the implantable system, for example wirelessly via the implantable transceiver 110.

In some embodiments, one or more of the following components may be contained in the implantable device 106: power source 112; memory 114; and a physiological data processing module 115.

The power source 112 may comprise a current source and/or a voltage source for providing the power for the signal delivered to a nerve by the electrode 108. The power source 112 may also provide power for the other components of the implantable device 106 and/or implantable system 116, such as the microprocessor 113, memory 114, and implantable transceiver 110. The power source 112 may comprise a battery, the battery may be rechargeable.

It will be appreciated that the availability of power is limited in implantable devices, and embodiments have been devised with this constraint in mind. The implantable device 106 and/or implantable system 116 may be powered by inductive powering or a rechargeable power source.

System Including Implantable Device

With reference to FIG. 8, the implantable device 106 of the disclosure may be part of a system 110 that includes a number of subsystems, for example the implantable system 116 and the external system 118. The external system 118 may be used for powering and programming the implantable system 116 and/or the implantable device 106 through human skin and underlying tissues.

The external subsystem 118 may comprise, in addition to controller 101, one or more of: a powering unit 102, for wirelessly recharging the battery of power source 112 used to power the implantable device 106; and, a programming unit 103 configured to communicate with the implantable transceiver 110. The programming unit 103 and the implantable transceiver 110 may form a communication subsystem. In some embodiments, powering unit 102 is housed together with programming unit 103. In other embodiments, they can be housed in separate devices.

The external subsystem 118 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the implantable device 106, and may be used in addition to the power transmission antenna 104 for transmitting an electromagnetic field at a high frequency (e.g., from 1 MHz to 10 GHz). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna(e) is a coil of wire with or without a ferrite core forming an inductor with a defined inductance. This inductor may be coupled with a resonating capacitor and a resistive loss to form the resonant circuit. The frequency is set to match that of the electromagnetic field generated by the power transmission antenna 105. A second antenna of the at least one antennae of implantable transceiver 110 can be used in implantable system 116 for data reception and transmission from/to the external system 118. If more than one antenna is used in the implantable system 116, these antennae are rotated 30 degrees from one another to achieve a better degree of power transfer efficiency during slight misalignment with the with power transmission antenna 104.

External system 118 may comprise one or more external body-worn physiological sensors 121 (not shown) to detect signals indicative of one or more physiological parameters. The signals may be transmitted to the implantable system 116 via the at least one antennae of implantable transceiver 110. Alternatively or additionally, the signals may be transmitted to the external system 116 and then to the implantable system 116 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and/or stored in memory 114 to operate the implantable system 116 in a closed-loop fashion. The physiological parameters of the subject determined via signals received from the external sensor 121 may be used in addition to alternatively to the physiological parameters determined via signals received from the implanted physiological sensor 111.

For example, in a particular embodiment a detector external to the implantable device may include an optical detector including a camera capable of imaging the eye and determining changes in physiological parameters, in particular the physiological parameters described above. As explained above, in response to the determination of one or more of these physiological parameters, the detector may trigger delivery of signal to a nerve by the electrode 108, or may modify the parameters of the signal being delivered or a signal to be delivered to a nerve by the electrode 108 in the future.

The system 100 may include a safety protection feature that discontinues the electrical stimulation of a nerve in the following exemplary events: abnormal operation of the implantable system 116 (e.g. overvoltage); abnormal readout from an implanted physiological sensor 111 (e.g. temperature increase of more than 2 degrees Celsius or excessively high or low electrical impedance at the electrode-tissue interface); abnormal readout from an external body-worn physiological sensor 121 (not shown); or abnormal response to stimulation detected by an operator (e.g. a physician or the subject). The safety precaution feature may be implemented via controller 101 and communicated to the implantable system 116, or internally within the implantable system 116.

The external system 118 may comprise an actuator 120 (not shown) which, upon being pressed by an operator (e.g. a physician or the subject), will deliver a signal, via controller 101 and the respective communication subsystem, to trigger the microprocessor 113 of the implantable system 116 to deliver a signal to the nerve by the electrode 108.

System 100 of the disclosure, including the external system 118, but in particular implantable system 116, can be made from, or coated with, a biostable and biocompatible material. This means that the device/system is both protected from damage due to exposure to the body's tissues and also minimizes the risk that the device/system elicits an unfavorable reaction by the host (which could ultimately lead to rejection). The material used to make or coat the device/system should ideally resist the formation of biofilms. Suitable materials include, but are not limited to, poly(p-xylylene) polymers (known as Parylenes) and polytetrafluoroethylene.

The implantable device 116 of embodiments of the disclosure will generally weigh less than 50 g.

General

The term “comprising” encompasses “including” as well as “consisting,” e.g. a composition “comprising” X may consist exclusively of X or may include something additional, e.g. X+Y.

The word “substantially” does not exclude “completely” e.g. a composition which is “substantially free” from Y may be completely free from Y. Where necessary, the word “substantially” may be omitted from definitions of the disclosure.

The term “about” in relation to a numerical value x is optional and means, for example, x+10%.

Unless otherwise indicated each embodiment as described herein may be combined with another embodiment as described herein.

The methods described herein may be performed by software in machine readable form on a tangible storage medium e.g. in the form of a computer program comprising computer program code means adapted to perform any of the methods described herein when the program is run on a computer and where the computer program may be embodied on a computer readable medium. Examples of tangible (or non-transitory) storage media include disks, thumb drives, memory cards etc and do not include propagated signals. The software can be suitable for execution on a parallel processor or a serial processor such that the method may be carried out in any suitable order, or simultaneously. This acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

It will be appreciated that the modules described herein may be implemented in hardware or in software. Furthermore, the modules may be implemented at various locations throughout the system.

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a DSP, programmable logic array, or the like.

Any range or device value given herein may be extended or altered without losing the effect sought, as will be apparent to the skilled person.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method blocks or elements identified, but that such blocks or elements do not comprise an exclusive list and a method or apparatus may contain additional blocks or elements.

The steps of the methods described herein may be carried out in any suitable order, or simultaneously where appropriate. Additionally, individual blocks may be deleted from any of the methods without departing from the spirit and scope of the subject matter described herein. Aspects of any of the examples described above may be combined with aspects of any of the other examples described to form further examples without losing the effect sought. Any of the modules described above may be implemented in hardware or software.

It will be understood that the above description of one embodiment is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the scope of this invention.

1. A nerve interface device comprising:
at least one cuff portion; and
a first pair of electrodes mounted on the at least one cuff portion;
wherein the at least one cuff portion has an assembled position in which the at least one cuff portion forms at least part of a passageway for receiving a nerve along a cuff axis passing through the passageway; and
wherein electrodes of the first pair are spaced apart from one another in a direction of the cuff axis passing through the passageway.

2. The device of claim 1, wherein at least one of:
the passageway has a diameter and the cuff axis passing through the passageway is perpendicular to the diameter and parallel with the passageway;
the cuff portion forms at least part of a tubular section for receiving the nerve; or
the cuff axis passing through the passageway is parallel with a depth of the tubular section.

3. (canceled)

4. (canceled)

5. The device of claim 1, wherein the first pair of electrodes are spaced apart from one another in a direction perpendicular to a diameter of the passageway and parallel with the passageway.

6. The device of claim 1, wherein the first pair of electrodes is arranged to provide an electrical channel using the nerve.

7. The device of claim 6, wherein the electrical channel is provided along a longitudinal axis of the nerve.

8. The device of claim 1, further comprising:
a plurality of pairs of electrodes mounted on the cuff portion;
wherein each of the electrodes in each of the plurality of pairs of electrodes are spaced apart from one another in the direction of the cuff axis passing through the passageway.

9. The device of any claim 8, comprising:
a first set of electrodes mounted on the at least one cuff portion; and
a second set of electrodes mounted on the at least one cuff portion;
wherein, in the assembled position, the electrodes of the first set of electrodes are spaced apart from one another in a direction about the cuff axis passing through the passageway; and
wherein, in the assembled position, the electrodes of second set of electrodes are spaced apart from one another in the same direction.

10. The device of claim 9, wherein a maximum number of electrodes in each set is half a circumference of the nerve divided by an average diameter of fascicles within the nerve.

11-13. (canceled)

14. The device of claim 9, wherein at least one of:
the first set of electrodes comprises a first electrode in a pair of electrodes, and the second set of electrodes comprises a second electrode in the same pair of electrodes;
the electrodes of at least one of the first set of electrodes or the second set of electrodes are spaced apart around a circumference of the at least one cuff portion in the assembled positioned; or
the electrodes of at least one of the first set or the second set are spaced apart in a ring about the cuff axis.

15-20. (canceled)

21. The device of claim 1, wherein the electrodes of the first pair of electrodes are spaced apart from one another according to at least one of:
along a length of the nerve; or
by a distance between 0.5 mm and 15 mm.

22. The device of claim 1, wherein each electrode of the first pair of electrodes has a surface of between 0.05 mm and 3 mm in length.

23-33. (canceled)

34. The device of claim 8, further comprising:
a first set of electrodes mounted in a ring around a first circumference of the at least one cuff portion in the assembled position; and
a second set of electrodes mounted in a ring around a second circumference of the at least one cuff portion in the assembled position;
wherein the first circumference is spaced apart from the second circumference in the direction of the at least one cuff axis, and each of the electrodes of the first set is aligned with one of the electrodes in the second set thus forming a plurality of pairs of electrodes defining a plurality of distinct electrical channels for stimulating the nerve along a length of the nerve, wherein the plurality of pairs of electrodes includes the first pair.

35. The device of claim 8, wherein each one of the plurality of pairs of electrodes is electrically isolated from the other pairs of electrodes, such that each pair of electrodes can be stimulated independently from one another.

36. The device of claim 34, wherein each electrode in the first set is a cathode, and each electrode in the second set is an anode.

37. A nerve stimulation system comprising:
a first nerve interface device according to claim 1; and
a stimulation device arranged to generate an electrical signal;
wherein the stimulation device is arranged for electrical communication with the first pair of electrodes of the first nerve interface device, to provide the electrical signal to the first pair of electrodes of the nerve interface device.

38. The nerve stimulation system according to claim 37, wherein the stimulation device is arranged to provide an electrical signal to each one of a plurality of pairs of electrodes independently from another electrical signal applied to the other pairs of the plurality of pairs of electrodes.

39. The nerve stimulation system of claim 37, wherein the electrical signal has at least one of:
a frequency of 1 Hz-50 Hz;
a predetermined pulse width;
a current amplitude of 100 ma-2000 mA;
a current of 500 μm;
a frequency of 1 Hz-50 Hz; or
a bipolar pulse form.

40-49. (canceled)

50. The nerve stimulation system of claim 37, further comprising a physiological sensor arranged to detect physiological activity in a subject.

51. The nerve stimulation system of claim 50, further comprising a comparison module arranged to detect a relationship between the electrical signal and the physiological activity.

52. A nerve stimulation method comprising:
providing a first nerve interface device according to claim 1; and
stimulating the first pair of electrodes of the first device, to provide an electrical signal to the first pair of electrodes of the first device.

53-64. (canceled)

65. A non-transitory computer-readable storage medium having stored thereon a computer program comprising code portions which, when loaded and run on a computing device, cause the computing device to stimulate the first pair of electrodes of the nerve interface device of claim 1, to provide an electrical signal to the first pair of electrodes.

66. (canceled)

67. An implantable system for at least one of stimulating or monitoring activity in a nerve, comprising:
at least one nerve interface device according to claim 1 arranged, in use, to apply an electrical signal to at least one nerve fiber of a subject;
a signal generator configured to generate a signal to be delivered to the at least one nerve fiber by the first pair of electrodes to modulate neural activity within the at least one nerve fiber; and
a control sub-system configured to cause the signal generator to deliver the signal to the first pair of electrodes.

68. The implantable system of claim 67, further comprising a detection sub-system configured to detect activity within the at least one nerve fiber.

69. (canceled)

70. The implantable system of claim 68, further comprising one or more physiological sensors configured to detect physiological activity that is associated with corresponding neural activity, within the at least one nerve fiber;
wherein the detection sub-system is configured to detect corresponding neural activity within the at least one nerve fiber at the first pair of electrodes.

71-79. (canceled)

80. The implantable system of claim 67, further comprising:
a first nerve interface device and a second nerve interface device, the first nerve interface device arranged, in use, to apply an electrical signal to at least one nerve fiber of a subject and the second nerve interface device arranged, in use, to detect the electrical signal in the at least one nerve fiber;
wherein the signal generator is configured to generate a signal to be delivered to the at least one nerve fiber by a first pair of electrodes in the first nerve interface device to modulate neural activity within the at least one nerve fiber;
wherein the control sub-system is configured to cause the signal generator to deliver the signal to the first pair of electrodes in the first nerve interface device; and
wherein the implantable system further comprises a detection sub-system configured to detect activity within the at least one nerve fiber at the first pair of electrodes in the second nerve interface device.

81. (canceled)

82. (canceled)
